(19) United States
(12) Patent Application Publication
(10) Pub. No.: US 2024/0058048 A1
(43) Pub. Date: Feb. 22, 2024

(54) TREATMENT TOOL GENERATOR, TREATMENT SYSTEM, CONTROL METHOD, AND TREATMENT METHOD

(71) Applicant: OLYMPUS CORPORATION, Tokyo (JP)

(72) Inventor: Shoei TSURUTA, Tokyo (JP)

(73) Assignee: OLYMPUS CORPORATION, Tokyo (JP)

(21) Appl. No.: 18/386,047

(22) Filed: Nov. 1, 2023

Related U.S. Application Data
(63) Continuation of application No. PCT/JP2021/033218, filed on Sep. 9, 2021.

(51) Int. Cl. A61B 18/12; A61B 1/00; A61B 18/14

(52) U.S. Cl. CPC A61B 18/1206; A61B 1/000094; A61B 18/1445; A61B 2018/00982

(57) ABSTRACT

A treatment tool generator includes: a power source configured to supply power to a treatment tool; and a processor configured to control an operation of the power source and the treatment tool, the processor being configured to cause the power source to supply the power to the treatment tool to apply treatment energy to a biological tissue from the treatment tool, calculate a thickness index value to be an index of a thickness dimension of the biological tissue that is grasped by the treatment tool, determine whether it is a change timing to change a control state of at least one of the power source and the treatment tool based on the thickness index value, and perform at least one of a first control, a second control, and a third control upon determining that it is the change timing.

### CROSS-REFERENCE TO RELATED APPLICATION

This application is a continuation of International Application No. PCT/JP2021/033218, filed on Sep. 9, 2021, the entire contents of which are incorporated herein by reference.

### BACKGROUND

### 1. Technical Field

The present disclosure relates to a treatment tool generator, a treatment system, a control method, and a treatment method.

### 2. Related Art

In the related art, a treatment tool that performs treatment of a biological tissue by applying treatment energy to the biological tissue according to supplied power has been known (for example, Japanese Patent No. 5198800).

### SUMMARY

In some embodiments, a treatment tool generator includes: a power source configured to supply power to a treatment tool; and a processor configured to control an operation of the power source and the treatment tool, the processor being configured to cause the power source to supply the power to the treatment tool to apply treatment energy to a biological tissue from the treatment tool, calculate a thickness index value to be an index of a thickness dimension of the biological tissue that is grasped by the treatment tool, determine whether it is a change timing to change a control state of at least one of the power source and the treatment tool based on the thickness index value, and perform at least one of a first control, a second control, and a third control upon determining that it is the change timing, the first control of reducing or suspending the power being supplied to the treatment tool from the power source for a predetermined time period, the second control of increasing a grasping force to grasp the biological tissue by the treatment tool, the third control of decreasing the grasping force to grasp the biological tissue by the treatment tool for a predetermined time period.

In some embodiments, a treatment tool generator includes: a power source configured to supply power to a treatment tool; and a processor configured to control an operation of the power source, the processor being configured to cause the power source to supply the power to the treatment tool to apply treatment energy to a biological tissue from the treatment tool, calculate a thickness index value to be an index of a thickness dimension of the biological tissue that is grasped by the treatment tool, determine whether it is a change timing to change a grasping state of the biological tissue by the treatment tool based on the thickness index value, and cause a notifier to notify information to prompt a change of the grasping state of the biological tissue upon determining that it is the change timing.

In some embodiments, a treatment system includes: a treatment tool; a treatment tool generator configured to control an operation of the treatment tool. The treatment tool generator includes a power source configured to supply power to the treatment tool; and a processor configured to control an operation of the power source and the treatment tool, and the processor is configured to cause the power source to supply the power to the treatment tool to apply treatment energy to a biological tissue from the treatment tool, calculate a thickness index value to be an index of a thickness dimension of the biological tissue that is grasped by the treatment tool, determine whether it is a change timing to change a control state of at least one of the power source and the treatment tool based on the thickness index value, and perform at least one of a first control, a second control, and a third control upon determining that it is the change timing, the first control of reducing or suspending the power being supplied to the treatment tool from the power source for a predetermined time period, the second control of increasing a grasping force to grasp the biological tissue by the treatment tool, the third control of decreasing the grasping force to grasp the biological tissue by the treatment tool for a predetermined time period.

In some embodiments, a treatment system includes: a treatment tool; and a treatment tool generator configured to control an operation of the treatment tool. The treatment tool generator includes a power source configured to supply power to the treatment tool; and a processor configured to control an operation of the power source, the processor is configured to cause the power source to supply the power to the treatment tool to apply treatment energy to a biological tissue from the treatment tool, calculate a thickness index value to be an index of a thickness dimension of the biological tissue that is grasped by the treatment tool, determine whether it is a change timing to change a grasping state of the biological tissue by the treatment tool based on the thickness index value, and cause a notifier to notify information to prompt a change of the grasping state of the biological tissue upon determining that it is the change timing.

In some embodiments, provided is a control method that is performed by a processor of a treatment tool generator. The method includes: supplying power to a treatment tool grasping a biological tissue from a power source to apply treatment energy to the biological tissue from the treatment tool; calculating a thickness index value to be an index value of a thickness dimension of the biological tissue grasped by the treatment tool; determining whether it is a change timing to change a control state of at least one of the power source and the treatment tool based on the thickness index value; and performing at least one of a first control, a second control, and a third control upon determining that it is the change timing, the first control of reducing or suspending the power being supplied to the treatment tool from the power source for a predetermined time period, the second control of increasing a grasping force to grasp the biological tissue by the treatment tool, the third control of decreasing the grasping force to grasp the biological tissue by the treatment tool for a predetermined time period.

In some embodiments, provided is a treatment method of treating a biological tissue by a treatment tool grasping the biological tissue. The method includes: causing, by a processor of a treatment tool generator, a power source to supply power to a treatment tool therefrom to apply treatment energy to the biological tissue from the treatment tool; calculating, by the processor, a thickness index value to be an index value of a thickness dimension of the biological tissue grasped by the treatment tool; determining, by the processor, whether it is a change timing to change a grasping state of the biological tissue by the treatment tool based on the thickness index value; and causing, by the processor, a notifier to notify information to prompt a change of the grasping state of the biological tissue upon determining that it is the changing timing.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment system according to a first embodiment;

FIG. 2 is a cross-section illustrating a configuration of a grasping portion;

FIG. 3 is a flowchart illustrating a control method performed by a second processor;

FIG. 4 is a diagram explaining the control method;

FIG. 5 is a diagram explaining the control method;

FIG. 6 is a diagram explaining step S4;

FIG. 7 is a diagram explaining a configuration of a treatment tool according to a second embodiment;

FIG. 8 is a flowchart illustrating a control method performed by the second processor;

FIG. 9 is a diagram explaining the control method;

FIG. 10 is a diagram explaining step S6;

FIG. 11 is a flowchart illustrating a control method performed by a second processor according to a third embodiment;

FIG. 12 is a diagram explaining a control method;

FIG. 13 is a diagram explaining a first modification of the first to the third embodiments;

FIG. 14 is a diagram explaining a second modification of the first to the third embodiments;

FIG. 15 is a diagram explaining a third modification of the first to the third embodiments;

FIG. 16 is a diagram explaining the third modification of the first to the third embodiments;

FIG. 17 is a diagram explaining the third modification of the first to the third embodiments;

FIG. 18 is a diagram explaining a conventional problem; and

FIG. 19 is a diagram explaining a conventional problem.

### DETAILED DESCRIPTION

Hereinafter, forms (hereinafter, embodiments) to implement the disclosure will be explained. the embodiments explained below are not intended to limit the disclosure. Furthermore, identical reference signs are assigned to identical parts throughout descriptions of the drawings.

### First Embodiment

Schematic Configuration of Treatment System

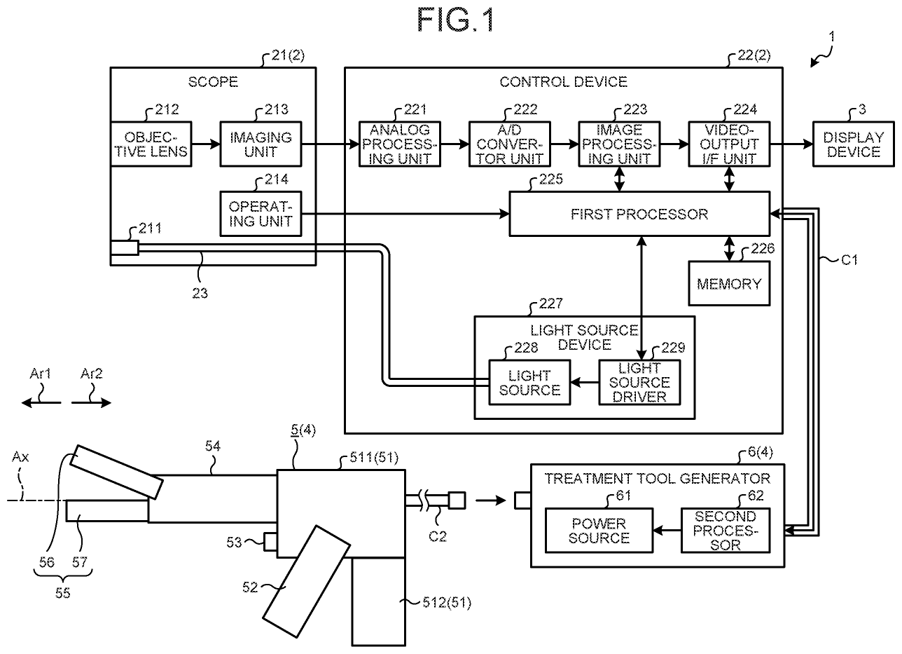

FIG. 1 is a diagram illustrating a treatment system 1 according to a first embodiment;

The treatment system 1 is a system to treat a biological tissue (hereinafter, denoted as target area), such as a blood vessel, to be treated inside a living body while observing the inside of the living body. This treatment system 1 includes, as illustrated in FIG. 1, an endoscope device 2, a display device 3, and a treatment device 4.

In the following, configurations of the endoscope device 2 and the treatment device 4 will be explained sequentially.

Configuration of Endoscope Device

The endoscope device 2 is a device that observes the inside of a living body. This endoscope device 2 includes, as illustrated in FIG. 1, a scope 21 and a control device 22.

The scope 21 is inserted into a living body, and images the inside of the living body. In the first embodiment, the scope 21 has a flexible thin long shape, and is constituted of a so-called flexible endoscope inserted into a living body, but it is not limited thereto, and it may be constituted of a so-called rigid endoscope. The scope 21 is detachably connected to the control device 22 through a connector (not illustrated). In this scope 21, as illustrated in FIG. 1, an illumination lens 211, an objective lens 212, an imaging unit 213, and an operating unit 214 are provided.

The illumination lens 211 is arranged at a distal end of the scope 21 facing toward an emitting end of a light guide 23 (FIG. 1). The light emitted from the light guide 23 passes through the illumination lens 211, and then irradiated to the inside of the living body.

The objective lens 212 is arranged at a distal end of the scope 21. The objective lens 212 takes in light (subject image) that has been irradiated to the inside of the living body from the illumination lens 211 and returned from the inside of the living body, and forms an image on a light receiving surface of the imaging unit 213.

The imaging unit 213 generates an endoscopic image by imaging the subject image formed by the objective lens 212 under control of the control device 22. The imaging unit 213 outputs the generated endoscopic image to the control device 22.

The operating unit 214 includes various kinds of switches (not illustrated) that accept user operations by a user, such as a doctor. The operating unit 214 outputs an operating signal according to the operation to the control device 22.

The control device 22 is constituted of a central processing unit (CPU), a field-programmable gate array (FPGA), and the like, and comprehensively controls operation of the scope 21 and the display device 3. This control device 22 includes, as illustrated in FIG. 1, an analog processing unit 221, an A/D converter unit 222, an image processing unit 223, a video-output I/F unit 224, a first processor 225, a memory 226, and a light source device 227.

The analog processing unit 221 receives an endoscopic image (analog signal) from the scope 21, and performs analog processing, such as clamp processing and noise removal processing (correlated double sampling (CDS)), with respect to the endoscopic image.

The A/D converter unit 222 A/D converts the endoscopic image (analog signal) subjected to the analog processing, and outputs the converted endoscopic image (digital signal).

The image processing unit 223 performs various kinds of image processing with respect to the input endoscopic image by using various kinds of parameters for image processing that are stored in the memory 226, under control of the first processor 225. Examples of the various kinds of image processing include optical black subtraction processing, white balance (WB) adjustment processing, demosaicing processing, color matrix operation processing, gamma correction processing, color reproduction processing, edge enhancement processing, and the like.

The video-output I/F unit 224 is constituted of a digital analog converter (DAC) encoder, or the like, and generates a video signal for display based on the endoscopic image (digital signal) subjected to various kinds of image processing by the image processing unit 223. The video-output I/F unit 224 outputs the video signal for display to the display device 3.

The display device 3 is constituted of a display that uses a liquid crystal or organic electroluminescence (EL), and the like. The display device 3 receives an input of the video signal for display from the video-output I/F unit 224, and displays the endoscopic image based on the video signal for display, and the like.

The light source device 227 includes, as illustrated in FIG. 1, a light source 228 and a light source driver 229. In the first embodiment, the light source device 227 is configured to be integrated in the control device 22, but it is not limited thereto, and may be configured independently of the control device 22.

The light source 228 is constituted of, for example, a white light emitting diode (LED) or the like, and emits light according to supplied power. The light emitted from the light source 228 passes through the light guide 23 and the illumination lens 211, and then irradiated to the inside of the living body.

The light source driver 229 supplies power to the light source 228 under control of the first processor 225.

The first processor 225 is constituted of, for example, a CPU and an FPGA, and controls operation of the scope 21, operation of the display device 3, and overall operation of the control device 22. Moreover, the control device 22 and a treatment tool generator 6 (FIG. 1) constituting the processing device 4 are detachably connected to each other through a first electric cable C1 (FIG. 1). The first processor 225 outputs an endoscopic image to the treatment tool generator 6 through the first electric cable C1.

The memory 226 stores a program executed by the first processor 225, information necessary for processing of the first processor 225, various kinds of parameters for the image processing described above, and the like.

Configuration of Treatment Device

The treatment device 4 applies treatment energy to a target area, and thereby treats the target area. The treatment enabled in the processing device 4 according to the first embodiment includes coagulation (hemostasis) and incision of the target area. Moreover, the treatment energy is high frequency energy. To apply high frequency energy to a target area means to flow high-frequency electric current to the respective target area. This processing device 4 includes, as illustrated in FIG. 1, a treatment tool 5 and the treatment tool generator 6.

The treatment tool 5 is a clamp-shaped treatment tool that grasps a target area, applies treatment energy to the target area, and thereby treats the target area. This treatment tool 5 includes, as illustrated in FIG. 1, a holding case 51, a movable handle 52, a switch 53, a shaft 54, and a grasping portion 55.

The holding case 51 supports the entire treatment tool 5. This holding case 51 includes, as illustrated in FIG. 1, a holding-case main body 511 and a fixed handle 512.

The holding-case main body 511 is positioned on the center axis Ax (FIG. 1) of the shaft 54, and has a substantially cylindrical shape extending along the center axis Ax.

The fixed handle 512 extends from the holding-case main body 511 toward a bottom side in FIG. 1, and is a portion grabbed by an operator.

The movable handle 52 is rotatably supported about an axis relative to the holding case 51, and receives a closing operation and an opening operation by the operator. The movable handle 52 rotates counterclockwise in FIG. 1 according to the closing operation by the operator, to come close to the fixed handle 512. Moreover, the movable handle 52 rotates clockwise in FIG. 1 according to the opening operation by the operator, to move away from the fixed handle 512.

The switch 53 is arranged in an exposed state to the outside from the holding case 51, and accepts a press (hereinafter, referred to as treatment start operation) by the operator. The switch 53 outputs an operating signal according to the treatment start operation to the treatment tool generator 6 through a second electric cable C2 (FIG.

As for the switch to accept the treatment start operation, not limited to a switch pressed by hand, a foot switch that is pressed by foot may be adopted.

The shaft 54 has a cylindrical shape, and is connected to the holding-case main body 511 at its end portion on a proximal end side (the right side in FIG. 1). Moreover, to an end portion on a distal end side (left side in FIG. 1) in the shaft 54, the grasping portion 55 is attached. Inside the shaft 54, an opening closing member 50 (refer to FIG. 4) that moves back and forth along the center axis Ax according to the closing operation and the opening operation performed by the operator with respect to the movable handle 52, and that opens and closes the first and the second grasping members 56, 57 (FIG. 1) constituting the grasping portion 55 is arranged. Depending on the treatment energy, the second grasping member 57 is not necessary to be substantially straight and, therefore, although a single-leaf opening and closing structure is illustrated as an example in FIG. 1, a double-leaf opening and closing structure may, of course, be adopted.

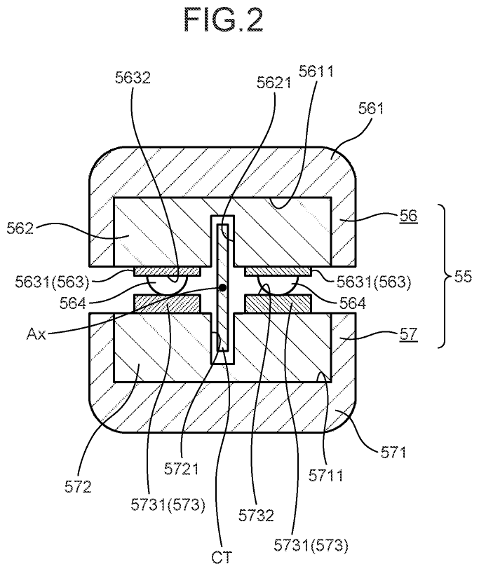

FIG. 2 is a cross-section illustrating a configuration of the grasping portion 55. Specifically, FIG. 2 is a cross-section of the grasping portion 55 cut along a plane perpendicular to the center axis Ax.

In the following, one side along the center axis Ax is denoted as distal end side Ar1 (FIG. 1), and the other side is denoted as proximal end side Ar2 (FIG. 1) for convenience of explanation.

The grasping portion 55 is a portion that grasps a target area, and that applies the treatment energy to the target area to thereby treat the target area. This grasping portion 55 includes the first and the second grasping members 56, 57 as illustrated in FIG. 1 or FIG. 2.

The first grasping member 56 includes, as illustrated in FIG. 2, a first jaw 561, a first supporting member 562, a first electrode 563, and an abutting portion 564.

The first jaw 561 is formed in a long shape extending along the center axis Ax. An end portion on the proximal end side Ar2 of the first jaw 561 is rotatably supported about a rotation axis extending in a direction perpendicular to a sheet surface of FIG. 1, with respect to an end portion on the distal end side Ar1 of the shaft 54. A portion of this first jaw 561 is constituted of a metal material, such as stainless steel and titanium to provide certain rigidity. The first jaw 561 (the first grasping member 56) opens and closes relative to the second grasping member 57 as the opening closing member 50 moves back and forth in the shaft 54 along the center axis Ax according to the closing operation and the opening operation performed with respect to the movable handle 52.

In this first jaw 561, on a surface on the second grasping member 57, a concave portion 5611 that is positioned at a central position in a width direction (the left and right direction in FIG. 2), and that extends along the center axis Ax is arranged as illustrated in FIG. 2.

The first supporting member 562 is a long flat plate that extends along the center axis Ax, and has an external shape substantially identical to an internal shape of the concave portion 5611. The first supporting member 562 fits in the concave portion 5611. The first supporting member 562 is constituted of an insulating material with low thermal conductivity, such as polytetrafluoroethylene (PTFE) and polyetheretherketone (PEEK). The first supporting member 562 is arranged between the first electrode 563 and the first jaw 561. That is, by arranging the first supporting member 562, the first jaw 561 and the first electrode 563 are electrically insulated from each other.

In the first supporting member 562, at a substantially central position in a width direction (the left and the right direction in FIG. 2) of a surface on the side closer to the second grasping member 57, as illustrated in FIG. 2, a cutter groove portion 5621 extending from a proximal end of the first supporting member 562 along the center axis Ax toward the distal end side Ar1 is arranged. However, when the jaw has a so-called Maryland-type structure in which the jaw bends in a direction different from an extension direction of the shaft, the position is not necessarily always be at the center axis Ax. At least, an electrode described later is present on both side in a width direction of the concave portion 5611.

The first electrode 563 is a portion to which high frequency power is supplied from the treatment tool generator 6 between itself and a second electrode 573 (FIG. 2) constituting the second grasping member 57. This first electrode 563 is constituted of a conductive material such as copper, and is a flat plate in a U-shape surrounding the cutter groove portion 5621 in a planar manner. The first electrode 563 is fixed to a surface on the side closer to the second grasping member 57 in the first supporting member 562 in such a position that both ends of the U-shape face the proximal end side Art. In FIG. 2, only a pair of extending portions 5631 that extend respectively along the center axis Ax are illustrated in the first electrode 563.

Furthermore, in the first electrode 563, a coating material (not illustrated) with non-adhesive properties to a living body is applied to a surface 5632 (FIG. 2) on the side closer to the second grasping member 57.

The abutting portion 564 has a hemisphere shape constituted of an insulating material, and is arranged on the surface 5632 in the first electrode 563. The abutting portion 564 abuts on the second electrode 573 when the first grasping material 56 closes with respect to the second grasping member 57. That is, the abutting portion 564 prevents a short circuit between the first and the second electrodes 563 and 573.

The second grasping member 57 includes, as illustrated in FIG. 2, a second jaw 571, a second supporting member 572, and a second electrode 573.

The second jaw 571 is a portion formed by extending a part of the shaft 54 toward the distal end side Ar1, and is formed in a long shape extending along the center axis Ax.

In this second jaw 571, on a surface on the side closer to the first grasping member 56, a concave portion 5711 that is positioned at a central position in a width direction (the left and right direction in FIG. 2), and that extends along the center axis Ax is arranged as illustrated in FIG. 2.

The second supporting member 572 is a long flat plate that extends along the center axis Ax, and has an external shape substantially identical to an internal shape of the concave portion 5711. The second supporting member 572 fits in the concave portion 5711. This second supporting member 572 is constituted of, for example, an insulating material with low thermal conductivity, such as PEEK. The second supporting member 572 is arranged between the second electrode 573 and the second jaw 571. That is, by arranging the second supporting member 572, the second jaw 571 and the second electrode 573 are electrically insulated from each other.

In the second supporting member 572, at a substantially central position in a width direction (the left and the right direction in FIG. 2) of a surface on the side closer to the first grasping member 56, as illustrated in FIG. 2, the cutter groove portion 5721 extending from a proximal end along the center axis Ax toward the distal end side Ar1 is arranged. This cutter groove portion 5721 opposes to the cutter groove portion 5621 when the first grasping member 56 is closed with respect to the second grasping member 57.

The second electrode 573 is a portion to which high frequency power is supplied between itself and the first electrode 563 by the treatment tool generator. This second electrode 573 is constituted of a conductive material, such as copper, and is a flat plate in a U-shape surrounding the cutter groove portion 5721 in a planar manner. The second electrode 573 is fixed to a surface on the side closer to the first grasping member 56 in the second supporting member 572 in such a position that both ends of the U-shape face the proximal end side Art. In FIG. 2, only a pair of extending portions 5731 that extend respectively along the center axis Ax are illustrated in the second electrode 573.

Moreover, in the second electrode 573, a coating material (not illustrated) with non-adhesive properties to a living body is applied to a surface 5732 (FIG. 2) on the side closer to the first grasping member 56.

Moreover, as illustrated in FIG. 2, a cutter CT that is positioned in the cutter groove portions 5621, 5721, and that moves back and forth along the center axis Ax according to an operation performed by an operator with respect to an operating lever (not illustrated) is arranged. That is, the target area grasped by the first and the second grasping members 56, 57 is incised by the back and forth movement of the cutter CT.

The treatment tool generator 6 is constituted of a CPU, an FPGA, and the like, and comprehensively controls movement of the treatment tool 5 connected through the second electric cable C2. This treatment tool generator 6 includes, as illustrated in FIG. 1, a power source 61 and a second processor 62.

The power source 61 supplies power (the high frequency power in the first embodiment) necessary to apply the treatment energy to the target area, to the treatment tool 5 connected through the second electric cable C2 under control of the second processor 62. Specifically, the power source 61 supplies high frequency power to a portion between the first and the second electrodes 563 and 573 through the second electric cable C2. Thus, to the first and the second electrodes 563 and 573, high frequency current flows. In other words, to the target area, high frequency energy is applied.

The second processor 62 corresponds to a processor. This second processor 62 is constituted of a CPU, an FPGA, and the like, and controls entire operation of the processing device 4 in accordance with a program stored in a memory (not illustrated).

Detailed functions of the second processor 62 will be explained in “Control Method Performed by Second Processor” described later.

Control Method Performed by Second Processor

Next, a control method performed by the second processor 62 will be explained.

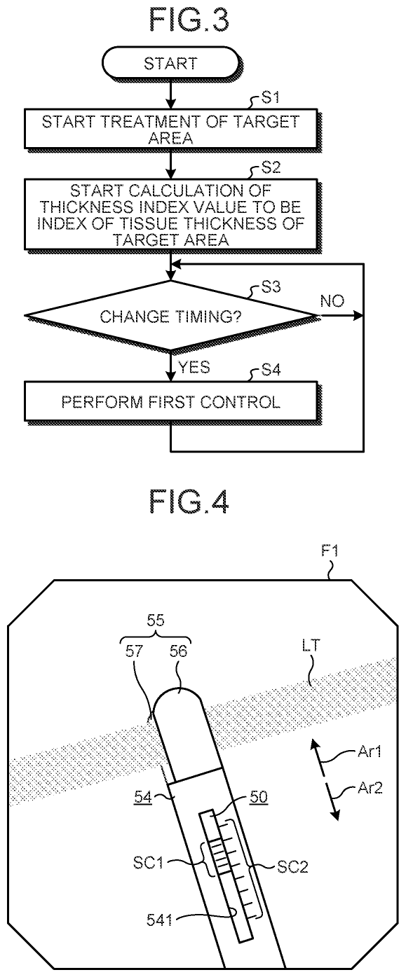

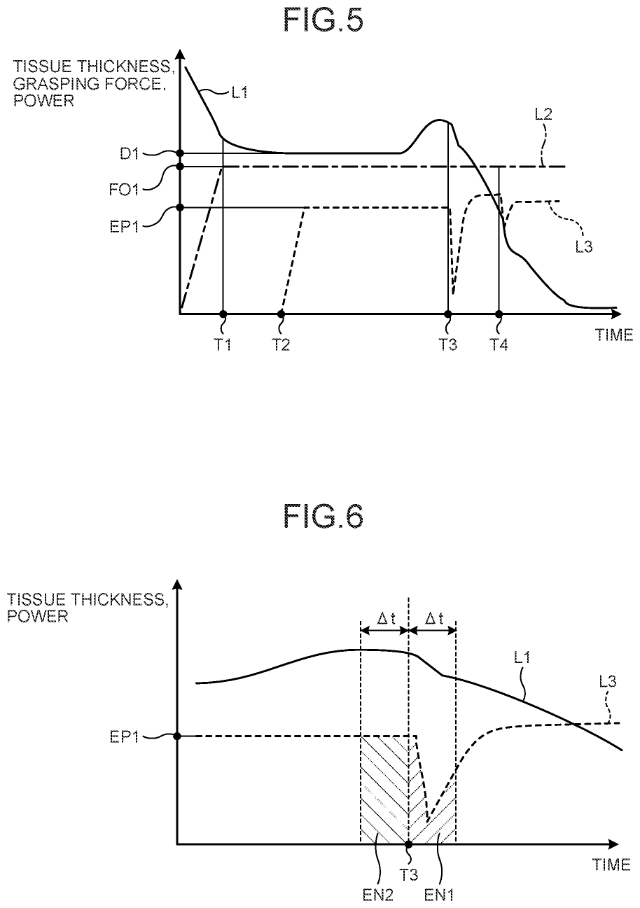

FIG. 3 is a flowchart illustrating the control method performed by the second processor 62. FIG. 4 and FIG. 5 are diagrams explaining the control method. Specifically, FIG. 4 is a diagram illustrating an endoscopic image F1 output to the treatment tool generator from the control device 22. FIG. 5 is a diagram respectively showing a behavior of a thickness dimension of a target area (hereinafter, referred to as tissue thickness) grasped between the first and the second grasping members 56 and 57, a behavior of a grasping force of grasping the target area with the first and the second grasping members 56 and 57, and a behavior of high frequency power supplied to the first and the second grasping members 56 and 57 from the power source 61. In FIG. 5, the behavior of the tissue thickness is represented by a line L1, the behavior of the grasping force is represented by a line L2, and the behavior of the high frequency power is represented by a line L3.

First, an operator holds the treatment tool 5 by hand, and performs the closing operation with respect to the movable handle 52, to thereby grasp a target area between the first and the second grasping members 56 and 57. For example, the movable handle 52 comes close to the fixed handle 512 according to the closing operation by the operator, and at the point when it is apart from the fixed handle 512 by a predetermined distance, a state of being apart from the fixed handle 512 by the predetermined distance is maintained by a ratchet mechanism or the like. Thus, the grasping of the target area is completed. In FIG. 5, the timing when the grasping of the target area is completed is a timing T1. That is, until it reaches the timing T, the grasping force gradually increases as indicated by the line L2 in FIG. 5. The grasping force becomes substantially constant at grasping force FO1 after the timing T1. Moreover, until it reaches the timing T1, the tissue thickness gradually decreases as indicated by the line L1 in FIG. 5. Because the grasped target area is a viscoelastic material, even with the same load, it gradually becomes thinner due to creep, but it is described herein as substantially constant state.

Next, the operator performs the treatment start operation with respect to the switch 53. In FIG. 5, the timing at which the treatment start operation is performed is a timing T2. Thus, the second processor 62 performs following control.

The second processor 62 starts treatment of the target area (step S1).

Specifically, the second processor 62 controls operation of the power source 61 to supply high frequency power to the first and the second electrodes 563, 573 from the power source 61 through the second electric cable C2. That is, the high frequency power increases after the timing T2 until it reaches high frequency power EP1, and becomes substantially constant at the high frequency power EP1 as indicated by the line L3 in FIG. 5.

After step S1, the second processor 62 starts calculation of a thickness index value to be an index of the tissue thickness (step S2).

Specifically, the second processor 62 calculates the thickness index based on the endoscopic image F1 output from the control device 22 through the first electric cable C1. The endoscopic image F1 is an image capturing a state in which the target area LT is grasped by the first and the second grasping members 56, 57 as illustrated in FIG. 4.

At an end portion on the distal end side Ar1 in the shaft 54, a through hole 541 that communicates the inside and the outside of the shaft 54, and that extends linearly along the center axis Ax is arranged as illustrated in FIG. 4. That is, the opening closing member 50 arranged inside the shaft 54 is visible through the through hole 541. Moreover, on the opening closing member 50 and a peripheral portion of the through hole 541, scales SC1, SC2 for recognizing an amount of back and forth movement of the opening closing member 50 in the shaft 54 are arranged. Because the first and the second grasping members 56 and 57 open and close according to the back and forth movement of the opening closing member 50, the back and forth movement has a correlation with a separation distance between the first and the second grasping members 56 and 57, in other words, with the tissue thickness. The second processor 62 recognizes an amount of the back and forth movement of the opening closing member 50 from the scales SC1, SC2 captured in the endoscopic image F1, and calculates the thickness index value to be an index of a tissue thickness based on the back and forth movement.

After step S2, the second processor 62 determines whether it is a change timing to change the control state of the power source 61 based on the thickness index value (step S3). In FIG. 5, the change timing is a timing T3.

Specifically, as the determination method at step S3, at least one determination method out of first and second determination methods described below is considered to be used.

The first determination method is a method of determining a point of time when a product of an amount of change in the thickness index value and a predetermined time period crosses a specific first threshold as the change timing. More specifically, the first determination method is a method of determining a point of time when a moving average of the amount of change in the thickness index value crosses the specific first threshold as the change timing.

Specifically, the moving average described above is expressed by following Equation 1 where a in infinitesimal change amount in a thickness index value Gap is dGap. The second processor 62 sequentially calculates the moving average of, for example, past 20 ms by Equation 1, and determines a point of time when the moving average becomes smaller than the first threshold as the change timing. As the first threshold, for example, −2 μm to −5 μn or the like can be considered.

Moving Average=∫dGapdt (1)

The method described above uses a simple moving average as a product of an amount of change in the thickness index value and the predetermined time period, but the disclosure is not limited thereto and, for example, a linear weighted moving average may be used.

Specifically, when the linear weighted moving average of past 20 ms is used with a control period of 1 ms, it is expressed by following Equation 2.

Linear Weighted Moving Average=(Value Before 20 ms×1+Value Before 19 ms×2+ . . . +Value Before N ms×(21−N)+ . . . +Value before 1 ms×20)÷210 (2)

The second determination method is a method of determining a point of time when a ratio of change dGAP/dt in the thickness index value Gap becomes lower than a second threshold as the change timing. As the second threshold, for example, −0.3 mm/s or the like is considered.

When it is determined that it is not the change timing (step S3: NO), the second processor 62 continues step S3 until it is determined as the change timing.

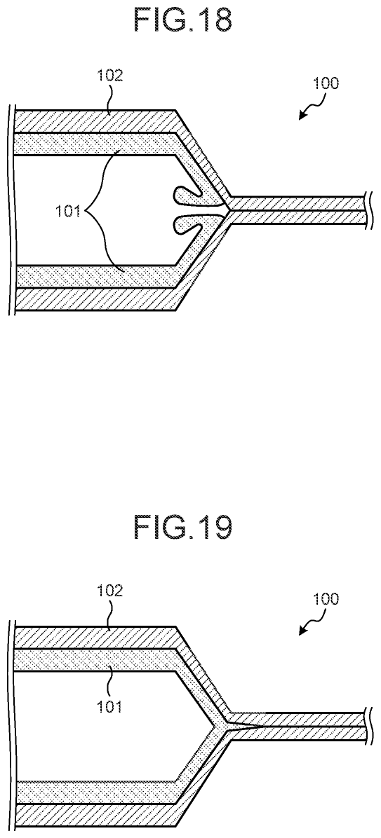

The behavior of the tissue thickness up until the change timing T3 shows, as indicated by the line L1 in FIG. 5, an expansion from a state of the tissue thickness D1 by application of the high frequency energy and a subsequent decrease. For example, the behavior of the tissue thickness means a state in which, if the target area LT is a blood vessel 100 (refer to FIG. 18, FIG. 19), steam has started increasing between a media 101 and an adventitia 102. If the high frequency energy is applied as previously even in this state, the steam increases explosively, and there is a risk that the media 101 and the adventitia 102 come apart from each other as illustrated in FIG. 18. When it is determined that it is the change timing (step S3: YES), the second processor 62 performs the first control (step S4). Thereafter, the second processor 62 returns to step S3.

FIG. 6 is a diagram explaining step S4. Specifically, FIG. 6 is an enlarged view of a part around the change timing T3 in FIG. 5.

Specifically, the second processor 62 controls operation of the power source 61 as the first control at step S4, and reduces the high frequency power being supplied to the first to the second electrodes 563 and 573 from the power source 61 temporarily for a predetermined time period from the high frequency power EP1, and then increases the high frequency power.

For example, the second processor 62 sets a power amount EN1 (expressed by oblique line hatching in FIG. 6) of the high frequency power in the predetermined time period Δt (for example, 0.15 s) just after the change timing T3 to be ½ to ⅓ of a power amount EN2 (expressed by oblique line hatching in FIG. 6) of the high frequency power in the predetermined time period Δt (for example, 0.15 s) just before the change timing T3, or smaller. The predetermined time period is not particularly limited and may be determined appropriately depending on a condition of a treatment site, but may be, for example, 5 to 50 ms.

It is noted that an application pattern of the high frequency power described above near the change timings T3 and T4 is not limited to the one described. That is, although it described as substantially constant with the high frequency power EP1 before the timing T3 in the above, it may be changed over time to increase gradually. The main focus of the present application is discontinuous changes in a power amount before and after the timing described above, and as long as a difference between the power amount EN1 and the power amount EN2 is within the range described above, the power application pattern is arbitrarily determined.

In the first control, as a power amount to increase the high frequency power after temporarily reducing the high frequency power, a power amount to compensate for a power amount of the high frequency power reduced as a result of temporary reduction can be considered as an example.

While an example in which the first control is performed at step S4 has been provided, a second control or a third control described later may be performed as step S4 in FIG. 3, or both the first control and the third control may be performed.

In an example in FIG. 5, a case in which the change timing is determined two times at step S3 by repeatedly performing steps S3 and S4. In FIG. 5, the second change timing is a timing T4.

The second processor 62 controls, when performing the first control at step S4, a reduction amount of the high frequency power at the first change timing T3 to be smaller than a reduction amount of the high frequency power at the second change timing T4 as illustrated in FIG. 5. In other words, when performing the first control at step S4, the second processor 62 decreases the reduction amount of the high frequency power as the thickness index value becomes smaller, based on the thickness index value.

According to the first embodiment explained above, following effects are produced.

In the treatment tool generator 6 according to the first embodiment, the second processor 62 calculates the thickness index value to be an index of the tissue thickness of the target area LT that is grasped between the first and the second grasping members 56 and 57. Moreover, the second processor 62 determines whether it is a change timing to change the control state of the power source 61 based on the thickness index value. Upon determining that it is the change timing, the second processor 62 performs the first control.

Therefore, for example, if the target area LT is the blood vessel 100 (refer to FIG. 18 and FIG. 19), by reducing the high frequency power after the change timing, it is possible to avoid explosive increase of steam generated between the media 101 and the adventitia 102. That is, it is possible to avoid the media 101 and the adventitia 102 from coming apart from each other.

Therefore, according to the treatment tool generator 6 according to the first embodiment, a biological tissue can be appropriately treated.

Moreover, in the treatment tool generator 6 according to the first embodiment, the second processor 62 determines whether it is the change timing by using at least one of the first and the second determination methods described above.

Therefore, it is possible to determine an appropriate timing before the steam described above explosively increases as the change timing.

Particularly, in the first determination method described above, a moving average is used. Therefore, even if the thickness index value is mistakenly calculated, it is not to be determined immediately erroneously as the change timing, and it is possible to determine the change timing appropriately. The method for improving the S/N ratio and the method of increasing accuracy in the calculation of thickness index value are not limited to the method of simple moving average described. Various kinds of methods of another linear weighted moving average and the like, or an anomaly detection method, such as detection of outliers such as k-nearest neighbors algorithm used in machine learning, detection of abnormal portions by training on normal variations, detection of a change point and an anomaly based on deviations from a prediction model, can be applied.

Moreover, in the treatment tool generator 6 according to the first embodiment, the second processor 62 calculates the thickness index value based on the endoscopic image F1.

Therefore, it is possible to calculate the thickness index value easily with a simple configuration.

Furthermore, in the treatment tool generator 6 according to the first embodiment, when the change timing is determined multiple times in chronological order, in the first control, the second processor 62 decreases an amount of reduction of the high frequency power at the change timing later in the chronological order among the multiple change timings. In other words, the second processor 62 decreases a reducing amount of the high frequency power as the thickness index value becomes smaller based on the thickness index value in the first control.

Therefore, compared to a case in which a fixed amount is used for the reduction amount of the high-frequency power, it is possible to shorten treatment time of the target area LT.

### Second Embodiment

Next, a second embodiment will be explained.

In the following explanation, identical reference signs are assigned to components identical to the first embodiment described above, and detailed explanation thereof will be omitted or simplified.

In the second embodiment, a configuration of the treatment tool 5 and functions of the second processor are different from the first embodiment described above.

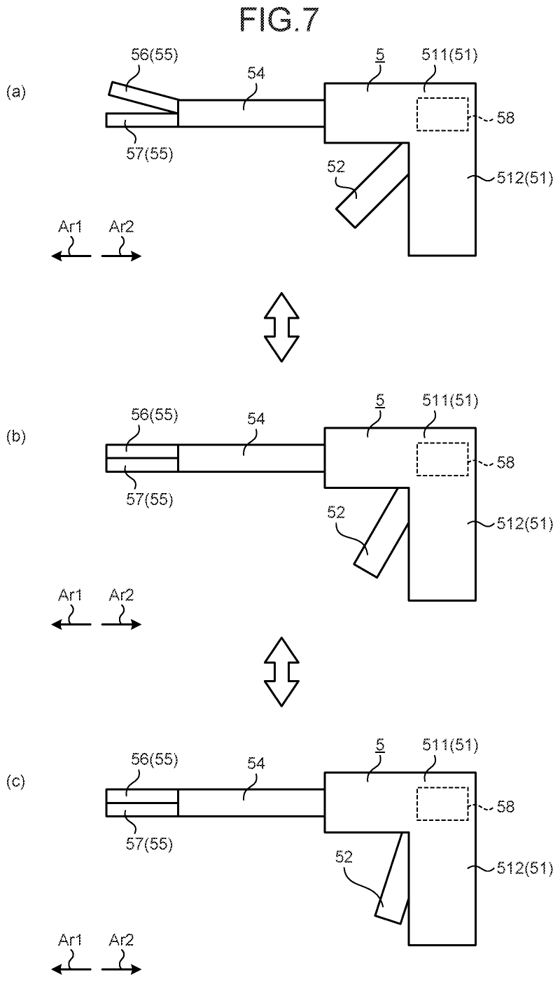

FIG. 7 is a diagram explaining a configuration of the treatment tool 5 according to the second embodiment.

In the treatment tool 5 according to the second embodiment, a motor 58 is added.

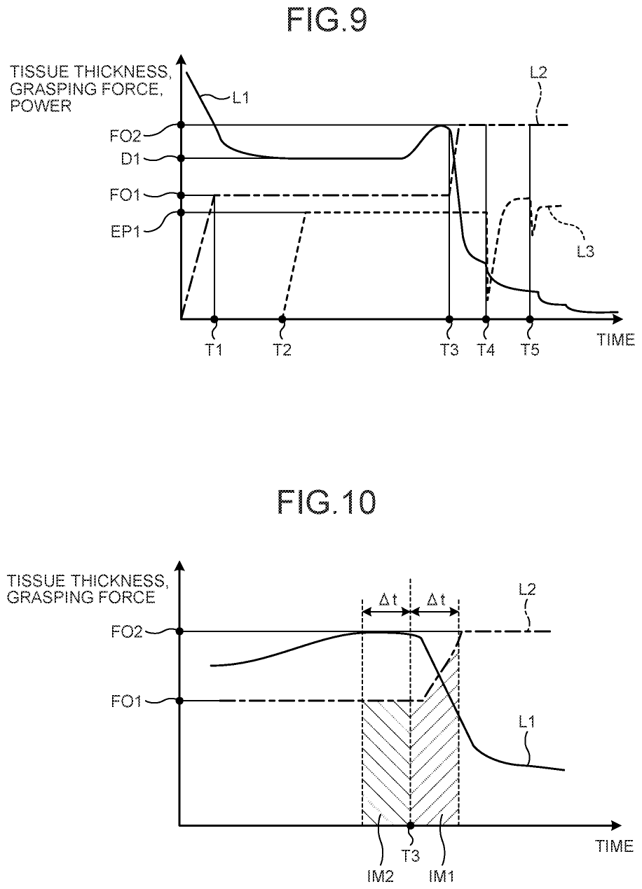

When the closing operation is performed by an operator from a state in which the first and the second grasping members 56 and 57 are farthest apart from each other ((a) in FIG. 7), the movable handle 52 comes close to the fixed handle 512, to be apart from the fixed handle 512 by a predetermined distance ((b) in FIG. 7). The movable handle 52 is restricted its movement in a direction separating from the fixed handle 512 by a ratchet mechanism at the point when it comes to be apart from the fixed handle 512 by the predetermined distance. In a state of (b) in FIG. 7 (hereinafter, denoted as first state), the first and the second grasping members 56 and 57 are in a closed state and is enabled to grasp the target area LT. In the first state, when the target area LT is grasped, the grasping force is the grasping force FO1 (refer to FIG. 9).

The motor 58 moves the opening closing member 50 back and forth inside the shaft 54 under control of the second processor 62, and brings the movable handle 52 closer ((c) in FIG. 7) to the fixed handle 512 from the first state illustrated in (b) in FIG. 7. That is, the motor 58 increases the grasping force to grasp the target area LT by the first and the second grasping members 56 and 57.

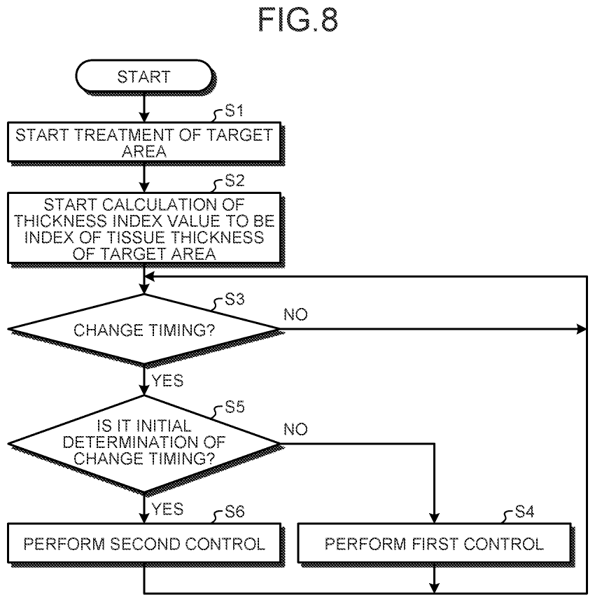

FIG. 8 is a flowchart illustrating a control method performed by the second processor 62. FIG. 9 is a diagram explaining the control method. Specifically, FIG. 9 is a diagram corresponding to FIG. 5.

Next, the functions of the second processor 62 according to the second embodiment will be explained, referring to FIG. 8 and FIG. 9.

In the control method performed by the second processor 62 according to the second embodiment, steps S5 and S6 are added to the control method explained in the first embodiment described above as illustrated in FIG. 8. Therefore, in the following, steps S5 and S6 are mainly explained.

The operator performs the closing operation with respect to the movable handle 52 before causing step S1 to be performed by performing the treatment start operation with respect to the switch 53, and thereby grasps the target area LT between the first and the second grasping members 56 and 57 while bringing it into the first state illustrated in (b) in FIG. 7 from the state illustrated in (a) in FIG. 7. Thus, the grasping force after the timing T1 at which the grasp of the target area LT is completed becomes substantially constant at the grasping force FO1 as illustrated in FIG. 9.

Step S5 is performed when it is determined that it is the change timing at step S3 (step S3: YES).

Specifically, the second processor 62 determines whether it is initial determination of the change timing at step S5.

When it is determined that it is the initial determination of the change timing (step S5: YES), the second processor 62 performs the second control (step S6). Thereafter, the second processor 62 returns to step S3.

FIG. 10 is a diagram explaining step S6. Specifically, FIG. 10 is an enlarged view of a part around the change timing T3 in FIG. 9.

Specifically, the second processor 62 controls operation of the motor 58 through the second electric cable C2 at step S6, to cause the back and forth movement of the opening closing member 50 inside the shaft 54, and brings the movable handle 52 closer to the fixed handle 512 ((c) in FIG. 7). The second processor 62 increases the grasping force from the grasping force FO1 to grasping force FO2 as illustrated in FIG. 9 or FIG. 10.

For example, the second processor 62 sets a grasping force product IM1 (grasping force×time, expressed by oblique line hatching in FIG. 10) of specific time Δt (for example, 0.15 s) just after the change timing T3 to be 1.3 times a grasping force product IM2 (expressed by oblique line hatching in FIG. 10) of specific time Δt (for example, 0.15 s) just before the change timing T3, or more.

On the other hand, when it is determined that the determination of the change timing is not the initial determination but determination performed the second time or later (step S5: NO), the second processor 62 performs step S4. Thereafter, the second processor 62 returns to step S3.

In the example in FIG. 9, a case in which the determination of the change timing is performed three times at step S3 by repeatedly performing steps S3 to S6 is illustrated. In FIG. 9, the second change timing is the timing T4. Moreover, the third change timing is a timing T5.

The second processor 62 decreases a reduction amount of the high frequency power at the third change timing T5 to be smaller than a reduction amount of the high frequency power at the second change timing T4 as illustrated in FIG. 9, when performing the first control at step S4. In other words, the second processor 62 decreases, when performing the first control at step S4, a reduction amount of the high frequency power as the thickness index value becomes smaller based on the thickness index value.

Steps S4 and S6 may be performed simultaneously at the initial time at the change timing T3.

In the treatment tool generator 6 according to the second embodiment explained above, after the change timing, it is possible to increase the boiling point of the target area LT by increasing the grasping force from the grasping force FO1 to the grasping force FO2. As a result, for example, when the target area LT is the blood vessel 100 (refer to FIG. 18, FIG. 19), it is possible to avoid explosive increase of steam generated between the media 101 and the adventitia 102.

Therefore, when the treatment tool generator 6 according to the second embodiment is adopted, effects similar to those of the first embodiment described above can be produced.

### Third Embodiment

Next, a third embodiment will be explained.

In the following explanation, identical reference signs are assigned to components identical to those of the second embodiment described above, and detailed explanation thereof will be omitted or simplified.

In the third embodiment, functions of the second processor 62 are different from the second embodiment described above.

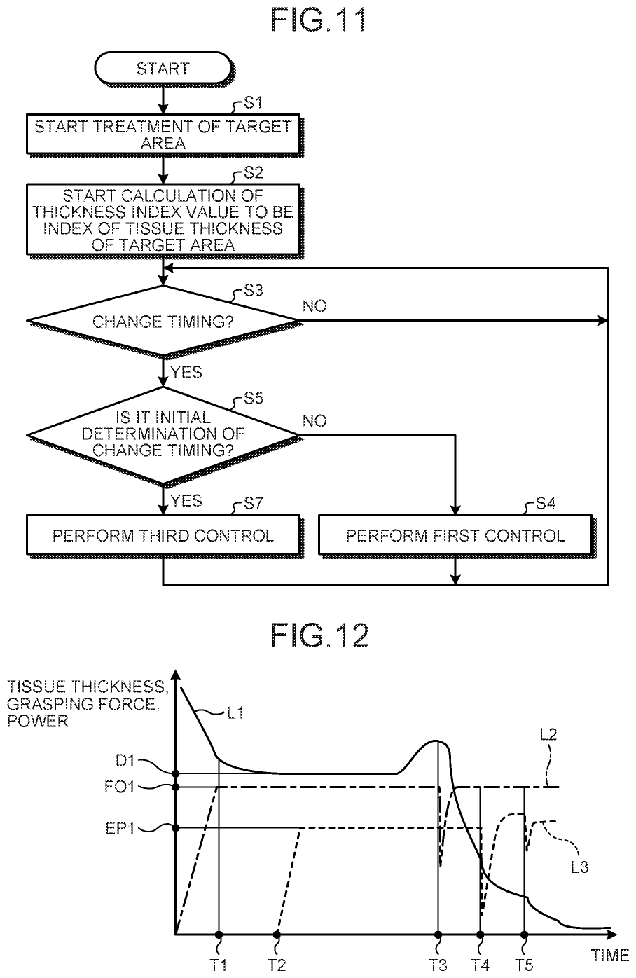

FIG. 11 is a flowchart illustrating a control method performed by the second processor 62 according to the third embodiment. FIG. 12 is a diagram explaining the control method. Specifically, FIG. 12 is a diagram corresponding to FIG. 9.

The functions of the second processor 62 according to the third embodiment will be explained, referring to FIG. 11 and FIG. 12.

In the control method performed by the second processor 62 according to the third embodiment, as illustrated in FIG. 11, step S7 is added in place of step S6 to the control method explained in the second embodiment described above. Therefore, in the following, step S7 is mainly explained.

Step S7 is performed when it is determined that the determination of the change timing is initial determination at step S5 (step S5: YES).

Specifically, the second processor 62 releases the restriction of movement of the movable handle 52 in the direction separating from the fixed handle 512 by the ratchet mechanism and the like at step S7. The second processor 62 controls operation of the motor 58, to cause the back and forth movement of the opening closing member 50 inside the shaft 54, and separates the movable handle 52 from the fixed handle 512, and increases the grasping force back to the grasping force FO1 after the third control in which the grasping force is temporarily reduced from the grasping force FO1. In the example in FIG. 12, after the grasping force is reduced temporarily from the grasping force FO1, it is increased back to the grasping force FO1.

Steps S4 and S7 may be performed simultaneously at the initial time at the change timing T3.

In the treatment tool generator 6 according to the third embodiment explained above, after the change timing, the grasping force is reduced from the grasping force FO1. As a result, for example, when the target area LT is the blood vessel 100 (refer to FIG. 18, FIG. 19), it is possible to avoid the media 101 and the adventitia 102 from coming apart from each other by steam generated between the media 101 and the adventitia 102.

Therefore, when the treatment tool generator 6 according to the third embodiment is adopted also, effects similar to those of the second embodiment described above are produced.

### Other Embodiments

The embodiments to implement the disclosure have so far been explained, but the disclosure is not to be limited to the first to the third embodiments described above.

In the first to the third embodiments described above, the scope 21 is constituted of a flexible endoscope, but it is not limited thereto. Instead of the scope 21, a rigid video scope, or a configuration in which a rigid endoscope and a camera head are combined may be adopted.

While the high frequency energy is adopted as the treatment energy to be applied to the target area LT in the first to the third embodiments described above, it is not limited thereto. As the treatment energy, at least either one of high frequency energy, ultrasound energy, and thermal energy can be adopted. To apply ultrasound energy to the target area LT means to apply ultrasound vibrations to the target area LT. Moreover, to apply thermal energy to the target area LT means to transfer heat generated by a heater or the like to the target area LT.

First Modification

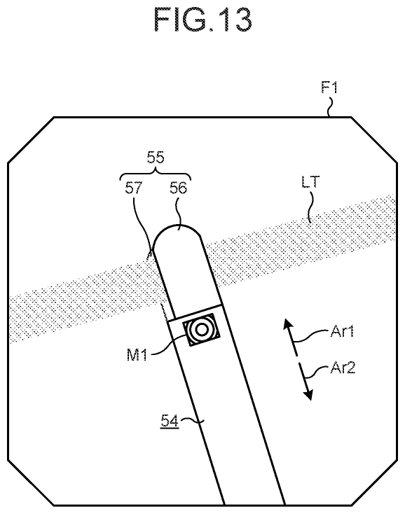

FIG. 13 is a diagram explaining a first modification of the first to the third embodiments. Specifically, FIG. 13 is a diagram corresponding to FIG. 4, and is a diagram illustrating the endoscopic image F1 output from the control device 22 to the treatment tool generator 6.

While the second processor 62 recognizes an amount of the back and forth movement of the opening closing member 50 from the scales SC1, SC2 captured in the endoscopic image F1, and calculates the thickness index value to be an index of a tissue thickness based on the back and forth movement in the first to the third embodiments described above, it is not limited thereto.

In the first modification, as illustrated in FIG. 13, the through hole 541 is not arranged at the end portion on the distal end side Ar1 of the shaft 54. Instead of that, a mark Ml to recognize interference fringes that changes in number depending on a separation distance between the first and the second grasping members 56 and 57 from the endoscopic image F1 is provided at the end portion on the distal end side Ar1. The second processor 62 calculates a separation distance between the first and the second grasping members 56 and 57, that is, the thickness index value to be an index of a tissue thickness, from the number of interference fringes at the mark Ml captured in the endoscopic image F1.

Moreover, the second processor 62 may calculate the thickness index value to be an index of a tissue thickness based on information captured in the endoscopic image F1, not limited to the amount of the back and forth movement of the opening closing member 50 explained in the first embodiment described above or the number of interference fringes explained in the first modification. For example, the second processor 62 may calculate the separation distance between the first and the second grasping members 56 and 57, that is, the thickness index value to be an index of a tissue thickness based on a positional relationship of the first and the second grasping members 56 and 57 captured in the endoscopic image F1.

Furthermore, a sensor to actually measure a tissue thickness may be arranged in the treatment tool 5, and a tissue thickness itself measured by the sensor may be used as the thickness index value.

Second Modification

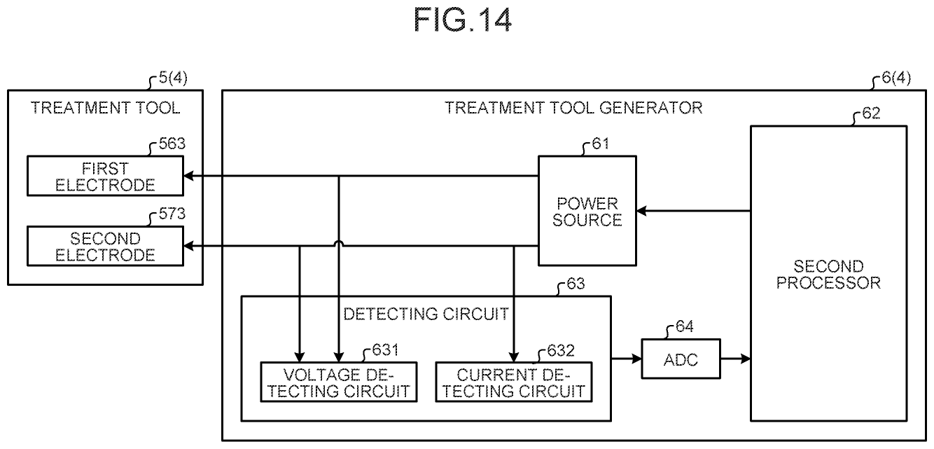

FIG. 14 is a diagram explaining a second modification of the first to the third embodiments. Specifically, FIG. 14 is a block diagram illustrating a configuration of the treatment tool generator 6 according to the second modification.

The second processor 62 calculates the thickness index value to be an index of a tissue thickness based on the endoscopic image F1 in the first to the third embodiments described above, but it is not limited thereto. For example, an impedance value of the target area LT may be used as the thickness index value.

Specifically, in the treatment tool generator 6 according to the second modification, as illustrated in FIG. 14, a detecting circuit 63 and an analog-to-digital converter (ADC) 64 are added to the treatment tool generator 6 explained in the first to the third embodiments described above.

The detecting circuit 63 includes a voltage detecting circuit 631 that detects a voltage value and a current detecting circuit 632 that detects a current value, and detects an HF signal according to high frequency power supplied to the first and the second electrodes 563 and 573 from the power source 61 over time.

Specifically, as examples of the HF signal, a high frequency current (hereinafter, denoted as HF current) and a high frequency voltage (hereinafter, denoted as HF voltage) supplied to the first and the second electrodes 563, 567 from the power source 61, high frequency power (hereinafter, denoted as HF power) calculated from the HF current and the HF voltage, an impedance value (hereinafter, denoted as HF impedance value) calculated from the HF current and the HF voltage, and the like can be provided.

The ADC 64 converts the HF signal (analog signal) output from the detecting circuit 63 into a digital signal. The ADC 64 then outputs the converted HF signal (digital signal) to the second processor 62.

The second processor 62 acquires the HF signal (HF impedance value) output from the ADC 64 as the thickness index value to be an index of a tissue thickness. The second processor 62 determines whether it is the change timing based on the HF impedance value.

For example, the second processor 62 determines a point of time when an absolute value of a change rate in the HF impedance value exceeds a specific threshold (for example, 100 Ω/s) as the change timing.

Moreover, for example, the second processor 62 sequentially calculates a difference between a current HF impedance value and an HF impedance value acquired immediately before, and determines a point of time when the difference rapidly becomes large as the change timing.

In this processing, although not particularly explained, the determination may be performed by using the moving average or various kinds of anomaly detection methods.

AS the thickness index value, not limited to the HF impedance value, the HF current or the HF voltage may be adopted. Alternatively, as the thickness index value, temperature of the target area LT after treatment is started, or viscosity of the target area LT may be adopted. A specific heat equivalent quantity and its temporal change when temperature is used, and a temporal change in viscosity when viscosity is used, and the like can be considered as an intermediate parameter for the determination.

Third Modification

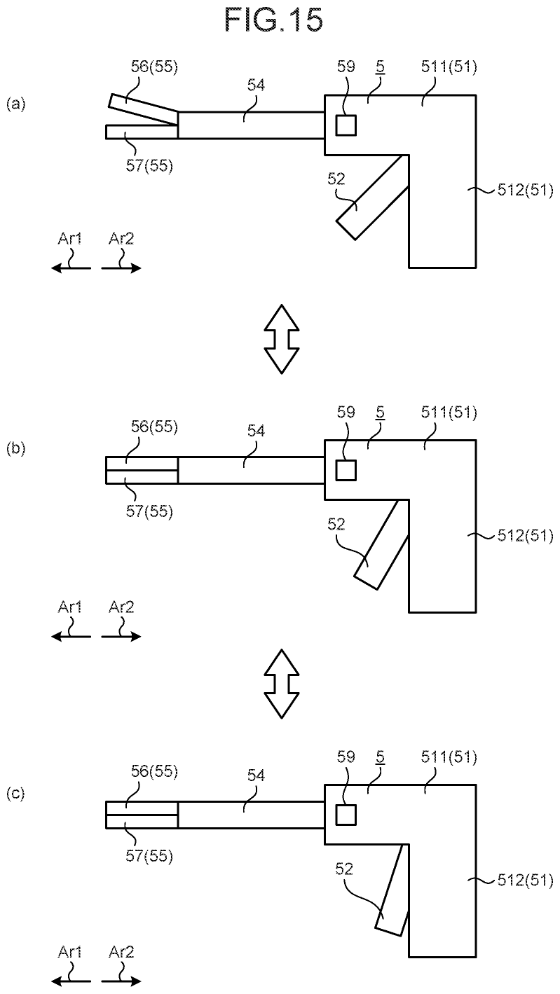

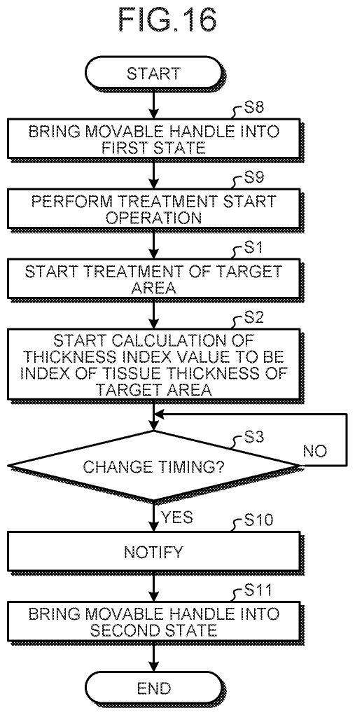

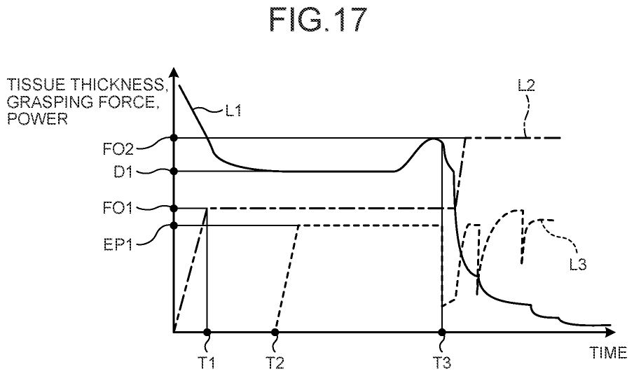

FIG. 15 to FIG. 17 are diagrams explaining a third modification of the second embodiment. Specifically, FIG. 15 is a diagram illustrating a configuration of the treatment tool 5 according to the third modification. FIG. 16 is a flowchart illustrating a treatment method according to the third modification. FIG. 17 is a diagram corresponding to FIG. 9.

In the second embodiment described above, the motor 58 is used when the grasping force is increased from the grasping force FO1 to the grasping force FO2 after determining that it is the change timing T3, but it is not limited thereto. As the third modification, it may be performed manually by an operator when increasing the grasping force from the grasping force FO1 to the grasping force FO2.

However, in this case, because modification of the grasping force is not performed automatically, there is a variable delay time from notification until a change in grasping force occurs. Therefore, a state in which the high frequency power is reduced is maintained for a predetermined time period. FIG. 17 illustrates an example thereof. Notification is performed at the timing T3, and subsequent energy treatment is then performed with a margin for changing the grasping force manually.

Specifically, for the predetermined time period, delay time of a fixed duration of time of, for example, 100 ms to 500 ms, is set, and a state in which the high frequency power is reduced is maintained. The high frequency power to be reduced may be gradually increased to avoid excessive reduction in tissue temperature, and it is not necessary to be constant. Moreover, completion of manual modification of the grasping force may be detected based on an endoscopic image or electrical information, and the state in which the high frequency power is reduced may be released based on the information as a trigger.

In the treatment tool 5 according to the third embodiment, the motor 58 is omitted from the treatment tool 5 according to the second embodiment described above, and a notifier 59 (FIG. 15) is added.

The notifier 59 notifies information to prompt a change of the grasping state of the target area LT under control of the second processor 62. As examples of this notifier 59, for example, a light emitting diode (LED) that notifies the information described above by lighting, flashing, or a color when it is lit, a display device that display the information described above, a speaker that outputs the information described above by a simple tone or sound, and the like can be provided. The notifier 59 may be arranged in the treatment tool 5 as illustrated in FIG. 15, or may be arranged in the treatment tool generator 6.

The movable handle 52 according to the third modification comes close to the fixed handle 512 when the closing operation is performed by an operator from a state in which the first and the second grasping members 56 and 57 are farthest apart from each other ((a) in FIG. 15) similarly to the second embodiment described above, to be apart from the fixed handle 512 by a first distance ((b) in FIG. 15). The movable handle 52 is restricted its movement in a direction separating from the fixed handle 512 by a ratchet mechanism or the like at the point when it comes to be apart from the fixed handle 512 by the first distance. In a state of (b) in FIG. 15 (hereinafter, denoted as first state), the first and the second grasping members 56 and 57 are in a closed state and is enabled to grasp the target area LT. In the first state, when the target area LT is grasped, the grasping force is the grasping force FO1.

Moreover, when the closing operation is performed by the operator from the first state, the movable handle 52 comes closer to the fixed handle 512, to be apart from the fixed handle 512 by a second distance that is shorter than the first distance ((c) in FIG. 15). The movable handle 52 is restricted its movement in a direction separating from the fixed handle 512 by the ratchet mechanism or the like at the point when it comes to be apart from the fixed handle 512 by the second distance. In the state of (c) in FIG. 15 (hereinafter, denoted as second state), if the target area LT is grasped, the grasping force is the grasping force FO2, which is larger than the grasping force FO1.

In the treatment method according to the third modification, as illustrated in FIG. 16, steps S8 to S11 are added to the control method explained in the second embodiment described above. Therefore, in the following, steps S8 to S11 will be mainly explained.

Steps S8 and S9 are performed prior to step S1.

Specifically, the operator performs the closing operation with respect to the movable handle 52 at step S8, and thereby grasps the target area LT between the first and the second grasping members 56 and 57 while bringing it into the first state illustrated in (b) in FIG. 15 from the state illustrated in (a) in FIG. 15. Thus, the grasping force after the timing T1 at which the grasp of the target area LT is completed becomes substantially constant at the grasping force FO1.

After step S8, the operator performs the treatment start operation with respect to the switch 53 (step S9). Thus, the second processor 62 performs processing at step S1 and later.

Step S10 is performed when it is determined as the change timing at step S3 (step S3: YES).

Specifically, the second processor 62 controls operation of the notifier 59 through the second electric cable C2 at step S10, and causes the notifier 59 to notify information to prompt a change of the grasping state of the target area LT.

After step S10, the operator recognizes the change timing to change the grasping state of the target area LT by the notification from the notifier 59. The operator then performs the closing operation with respect to the movable handle 52, to bring it into the second state illustrated in (c) in FIG. 15 from the first state (step S11). Thus, the grasping force increases to the grasping force FO2, which is larger than the grasping force FO1.

In the third embodiment described above, similarly to the third modification, the operator may manually reduce the grasping force from the grasping force FO1 in response to the notification from the notifier 59 at the change timing T3.

Although the embodiments and the modifications thereof described above are all described assuming a treatment tool that includes at least a portion that opens and closes the treatment tool manually, embodiments of the present application are not limited thereto. For example, it is needless to say that it is applicable to a robotic treatment tool that performs grasping operation entirely by the motor 58 without distinction between the fixed handle 512 and the movable handle 52 also.

According to the treatment tool generator, the treatment system, the control method, and the treatment method according to the disclosure, a biological tissue can be treated appropriately.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment tool generator comprising:
a power source configured to supply power to a treatment tool; and
a processor configured to control an operation of the power source and the treatment tool, the processor being configured to
cause the power source to supply the power to the treatment tool to apply treatment energy to a biological tissue from the treatment tool,
calculate a thickness index value to be an index of a thickness dimension of the biological tissue that is grasped by the treatment tool,
determine whether it is a change timing to change a control state of at least one of the power source and the treatment tool based on the thickness index value, and
perform at least one of a first control, a second control, and a third control upon determining that it is the change timing, the first control of reducing or suspending the power being supplied to the treatment tool from the power source for a predetermined time period, the second control of increasing a grasping force to grasp the biological tissue by the treatment tool, the third control of decreasing the grasping force to grasp the biological tissue by the treatment tool for a predetermined time period.

2. The treatment tool generator according to claim 1, wherein
the processor is configured to increase the power after temporarily reducing or suspending the power for the predetermined time period in the first control.

3. The treatment tool generator according to claim 1, wherein
the processor is configured to increase the grasping force after temporarily decreasing the grasping force for the predetermined time period in the third control.

4. The treatment tool generator according to claim 1, wherein
the processor is configured to determine, as the change timing, a point of time when a product of a change amount in the thickness index value and a predetermined time period crosses a specific first threshold.

5. The treatment tool generator according to claim 1, wherein
the processor is configured to determine, as the change timing, a point of time when a change rate of the thickness index value becomes lower than a second threshold.

6. The treatment tool generator according to claim 1, wherein
the processor is configured to acquire, from an endoscope device configured to generate an endoscopic image capturing a state in which the biological tissue is grasped by the treatment tool, the endoscopic image, and calculate the thickness index value based on the endoscopic image.

7. The treatment tool generator according to claim 1, wherein
the thickness index value is a value that is calculated from at least one of electric current, voltage, phase information of the current, and phase information of the voltage, the current and the voltage being applied as the power.

8. The treatment tool generator according to claim 1, wherein
the processor is configured to change a reduction amount of the power based on the thickness index value in the first control.

9. The treatment tool generator according to claim 1, wherein
when the change timing is determined a plurality of times chronologically, the processor is configured to decrease a reduction amount of the power at the change timing that is later in chronological order in a plurality of change timings determined at the plurality of times, in the first control.

10. A treatment tool generator comprising:
a power source configured to supply power to a treatment tool; and
a processor configured to control an operation of the power source, the processor being configured to
cause the power source to supply the power to the treatment tool to apply treatment energy to a biological tissue from the treatment tool,
calculate a thickness index value to be an index of a thickness dimension of the biological tissue that is grasped by the treatment tool,
determine whether it is a change timing to change a grasping state of the biological tissue by the treatment tool based on the thickness index value, and
cause a notifier to notify information to prompt a change of the grasping state of the biological tissue upon determining that it is the change timing.

11. A treatment system comprising:
a treatment tool;
a treatment tool generator configured to control an operation of the treatment tool, wherein
the treatment tool generator includes
a power source configured to supply power to the treatment tool; and
a processor configured to control an operation of the power source and the treatment tool, and
the processor is configured to
cause the power source to supply the power to the treatment tool to apply treatment energy to a biological tissue from the treatment tool,
calculate a thickness index value to be an index of a thickness dimension of the biological tissue that is grasped by the treatment tool,
determine whether it is a change timing to change a control state of at least one of the power source and the treatment tool based on the thickness index value, and
perform at least one of a first control, a second control, and a third control upon determining that it is the change timing, the first control of reducing or suspending the power being supplied to the treatment tool from the power source for a predetermined time period, the second control of increasing a grasping force to grasp the biological tissue by the treatment tool, the third control of decreasing the grasping force to grasp the biological tissue by the treatment tool for a predetermined time period.

12. A treatment system comprising:
a treatment tool; and
a treatment tool generator configured to control an operation of the treatment tool, wherein
the treatment tool generator includes
a power source configured to supply power to the treatment tool; and
a processor configured to control an operation of the power source,
the processor is configured to
cause the power source to supply the power to the treatment tool to apply treatment energy to a biological tissue from the treatment tool,
calculate a thickness index value to be an index of a thickness dimension of the biological tissue that is grasped by the treatment tool,
determine whether it is a change timing to change a grasping state of the biological tissue by the treatment tool based on the thickness index value, and
cause a notifier to notify information to prompt a change of the grasping state of the biological tissue upon determining that it is the change timing.

13. A control method that is performed by a processor of a treatment tool generator, the method comprising:
supplying power to a treatment tool grasping a biological tissue from a power source to apply treatment energy to the biological tissue from the treatment tool;
calculating a thickness index value to be an index value of a thickness dimension of the biological tissue grasped by the treatment tool;
determining whether it is a change timing to change a control state of at least one of the power source and the treatment tool based on the thickness index value; and
performing at least one of a first control, a second control, and a third control upon determining that it is the change timing, the first control of reducing or suspending the power being supplied to the treatment tool from the power source for a predetermined time period, the second control of increasing a grasping force to grasp the biological tissue by the treatment tool, the third control of decreasing the grasping force to grasp the biological tissue by the treatment tool for a predetermined time period.

14. A treatment method of treating a biological tissue by a treatment tool grasping the biological tissue, the method comprising:
causing, by a processor of a treatment tool generator, a power source to supply power to a treatment tool therefrom to apply treatment energy to the biological tissue from the treatment tool;
calculating, by the processor, a thickness index value to be an index value of a thickness dimension of the biological tissue grasped by the treatment tool;
determining, by the processor, whether it is a change timing to change a grasping state of the biological tissue by the treatment tool based on the thickness index value; and
causing, by the processor, a notifier to notify information to prompt a change of the grasping state of the biological tissue upon determining that it is the changing timing.

15. The treatment method according to claim 14, further comprising
changing, by an operator operating the treatment tool changes, a grasping force of the biological tissue by the treatment tool upon recognizing the information.
